(19) United States
(12) Patent Application Publication
(10) Pub. No.: US 2026/0315661 A1
(43) Pub. Date: Oct. 15, 2026

(54) SODIUM ALGINATE AND HYALURONIC ACID BLENDED HYDROGEL MATRIX

(71) Applicant: ALAFAIR BIOSCIENCES, INC., Austin, TX (US)

(72) Inventors: Vidhi Maheshwari, Austin, TX (US); Sarah Mayes, Austin, TX (US)

(21) Appl. No.: 19/678,294

(22) Filed: May 15, 2026

Related U.S. Application Data
(62) Division of application No. 19/278,963, filed on Jul. 24, 2025.
(60) Provisional application No. 63/676,696, filed on Jul. 29, 2024.

(51) Int. Cl. A61L 27/52; A61L 27/26; C08L 5/04; C08L 5/08

(52) U.S. Cl. CPC A61L 27/52; A61L 27/26; C08L 5/04; C08L 5/08; A61L 2300/236; A61L 2300/80; A61L 2400/06

(57) ABSTRACT

A biocompatible lyophilized pellet comprised of alginate and hyaluronate. The pellet may be used to provide a gliding surface for tissues impacted by surgery. The pellet may be delivered in a broad range of volumes to address large to micron-sized target tissues. The pellet may be hydrated before or after application. An external stimulus comprising a crosslinker may be applied to mold the pellet in situ around complex tissue geometries or to tailor the rate of bioresorption.

### CROSS-REFERENCE TO RELATED APPLICATIONS

This application is a divisional of U.S. patent application Ser. No. 19/278,963, filed Jul. 24, 2025, which claims priority to U.S. Provisional Patent Application No. 63/676,696 filed Jul. 29, 2024 and entitled “Sodium Alginate And Hyaluronic Acid Blended Hydrogel Matrix”. The content of each of the above applications is hereby incorporated by reference.

### TECHNICAL FIELD

An embodiment relates generally to the field of medical devices and more particularly to devices that allow tissue gliding. An embodiment is a bioresorbable lyophilized pellet that can be quickly rehydrated before or after insertion into the body, between tissues and organs to allow tissues to glide. The hydrated pellet can be delivered in aliquot volumes ranging from μL to mL and can be molded in situ via subsequent crosslinking with an external stimulus. This crosslinking may be used to tailor the shape and bioresorption rate of the implanted hydrogel.

### BACKGROUND

Tissue gliding is required for function. Postoperative scar tissue attachments, also called adhesions, are a frequent complication of surgical procedures that form because of reduced tissue gliding experienced during healing. During a surgical procedure the tissues and organs of the body may be deliberately or inadvertently injured. These injuries prompt a wound healing response that results in edema, increased friction between tissues, and decreased gliding. This decreased gliding exacerbates the injury response, often causing the body to scar together tissues that should remain separate. The timeline for this cascade of events varies from one area of the body to another. For instance, early-stage healing may be on the order of weeks for tendon, but on the order of months for peripheral nerve tissue. These adhesions can form on the submicron scale between challenging-to-reach or -to-visualize tissues such as between a tendon and tendon sheath. Adhesions can form in any anatomical location including around the eyes, tendons, ligaments, heart, spinal cord, peripheral nerve, sinus, and among the organs of the abdominopelvic cavities. Decreased gliding can produce pain and discomfort for the patient, impair the function of the effected tissues, and complicate subsequent surgeries in the same anatomical region.

Tissue protectors are medical devices used by surgeons to prevent tethering by allowing tissues to glide. These devices are commercially available as membranes, gels, and solutions. During a surgical procedure a surgeon may choose to insert a tissue protector between injured tissues and organs. Such devices work by reducing the coefficient of friction between injured organs during the critical period of wound healing during which patients are most at risk for adhesions. After the critical phase has passed the devices are resorbed by the body.

### BRIEF DESCRIPTION OF DRAWINGS

Features and advantages of embodiments of the present invention will become apparent from the appended claims, the following detailed description of one or more example embodiments, and the corresponding figures. Where considered appropriate, reference labels have been repeated among the figures to indicate corresponding or analogous elements.

FIG. 1 depicts an embodiment of a method for preparation of lyophilized HA/SA hydrogel.

FIGS. 2A-C depict an effect of freezing conditions on the structure of lyophilized pellets. FIG. 2A: Rigid pellet from a slow cooling rate (8 hours), FIGS. 2B-C: Layered or lamellar pellet from a rapid cooling rate (4 hours).

FIG. 3 shows test samples: pre-lyophilization, hydrated layered, and hydrated rigid.

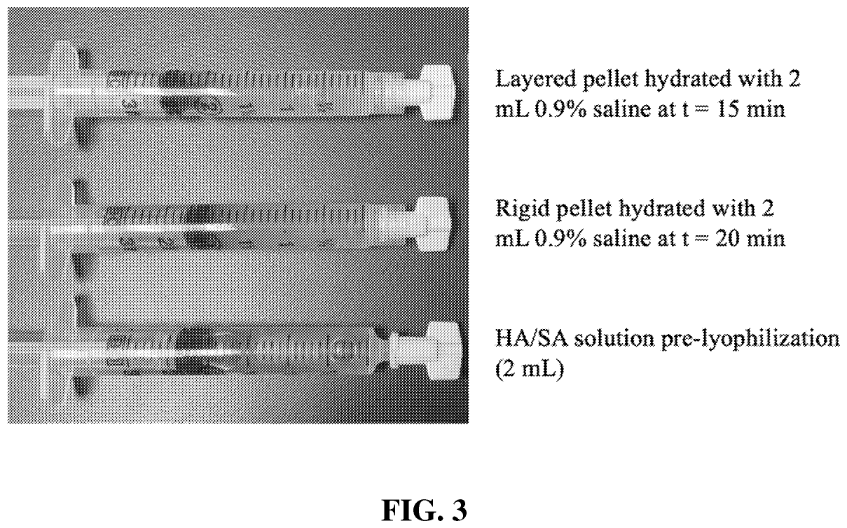

FIG. 4 provides a table for hydration time and resulting viscosity of rigid vs layered lyophilized HA/SA pellets vs. pre-frozen HA/SA solution.

FIG. 5 addresses pore size and distribution in a lyophilized HA/SA hydrogel pellets.

FIGS. 6A-E provide images of HA/SA pellets prepared with slow rate of cooling, over a period of 8 hours. FIG. 6A: Macroscopic image, Optical microscopy image; FIG. 6B: 20×; FIG. 6C: 200×, SEM; FIG. 6D: surface imaged at 500×; FIG. 6E: cross-section imaged at 500×; FIGS. 6F-J provide images of HA/SA pellets prepared with rapid rate of cooling, over a period of 4 hours. FIG. 6F: Macroscopic image, Optical microscopy image; FIG. 6G: 20 ×; FIG. 6H: 200×, SEM; FIG. 6I: surfaced imaged at 500×; FIG. 6J: cross-section imaged at 500×.

FIGS. 7A-C depict surface analysis via optical microscopy of a rigid lyophilized HA/SA pellet that revealed a planar structure. FIG. 7A: ROI at 200×magnification, FIG. 7B: 3-D contour map (Note: red (area 411)-to-blue (area 412) indicates depth into the pellet sample); FIG. 7C: Height map along the surface.

FIGS. 8A-C depict surface analysis via optical microscopy of a lamellar lyophilized HA/SA pellet that revealed parallel layers up to 1500 μm deep. FIG. 8A: ROI at 200× magnification, FIG. 8B: 3-D contour map (Note: red (area 401)-to-blue (area 402) indicates depth into the pellet sample), FIG. 8C: Height map along the surface

FIG. 9 provides a table for mechanical characterization of rigid and layered HA/SA hydrogel pellets.

FIG. 10 addresses mechanical characterization of rigid and layered HA/SA hydrogel pellets (* denotes p-value<0.05).

FIG. 11 addresses in situ cross-linking of HA/SA solution for tissue protection or mold formation.

FIG. 12 depicts a frusto-conical shaped hydrogel embodiment having a void to facilitate, for example, re-hydration.

### DETAILED DESCRIPTION

Reference will now be made to the drawings wherein like structures may be provided with like suffix reference designations. In order to show the structures of various embodiments more clearly, the drawings included herein are diagrammatic representations of structures. Thus, the actual appearance of the fabricated structures, for example in a photo, may appear different while still incorporating the claimed structures of the illustrated embodiments (e.g., walls may not be exactly orthogonal to one another in actual fabricated devices). Moreover, the drawings may only show the structures useful to understand the illustrated embodiments. Additional structures known in the art may not have been included to maintain the clarity of the drawings. For example, not every layer of a device is necessarily shown. “An embodiment”, “various embodiments” and the like indicate embodiment(s) so described may include particular features, structures, or characteristics, but not every embodiment necessarily includes the particular features, structures, or characteristics. Some embodiments may have some, all, or none of the features described for other embodiments. “First”, “second”, “third” and the like describe a common object and indicate different instances of like objects are being referred to. A “First” instance of a feature does necessarily mean there is a “Second” instance of the feature. Such adjectives do not imply objects so described must be in a given sequence, either temporally, spatially, in ranking, or in any other manner. “Connected” may indicate elements are in direct physical or electrical contact with each other and “coupled” may indicate elements co-operate or interact with each other, but they may or may not be in direct physical or electrical contact. Phrases such as “comprising at least one of A or B” include situations with A, B, or A and B.

Applicant determined in some cases tissue protectors not containing collagen are superior to collagen-based devices which are bioresorbed via remodeling into additional tissue, which can add bulk and introduce issues such as nerve compression that require removal. Many of even the most successful of commercially available protectors are only partially effective because of inability to reach all areas where adhesions form, inability to maintain a desired shape, inability to remain in place, or to have a tailored bioresorption rate. Applicant determined it would be advantageous clinically to provide collagen free tissue protectors capable of addressing tissue gliding in confined, challenging-to-reach places and the ability to tailor the shape and bioresorption rate of the device. Applicant further determined a tissue protector deliverable in flexible volumes, capable of reducing the coefficient of friction between challenging-to-reach tissues would meet this clinical need.

An embodiment provides a hydrogel pellet that can be quickly hydrated in an operating room setting, can be aliquoted onto target tissues in flexible volumes ranging from μL to mL, can be molded in situ to retain the shape of unique geometries, can remain in place during the desired period, can resorb without replacement, and is effective at reducing the coefficient of friction between tissues where placed.

One aspect of an embodiment is a lyophilized pellet comprising essentially alginate and hyaluronate. The pellets can be obtained by methods such as casting into a mold, spin-coating, doctor blading, aliquoting and the like, followed by flash freezing and drying under vacuum. The pellets are stabilized through lyophilization.

Another aspect of an embodiment is a method for using the aforementioned pellet to reduce the coefficient of friction between tissues. The pellet may be used in any anatomical location of the body for which there is a risk of scar tissue attachments. During a surgical procedure the lyophilized pellet is quickly hydrated with biological fluids, saline, or a therapeutic solution and then may be aliquoted in volumes ranging μL to mL scale onto or between two opposing organs or tissues. The aliquots may then be molded in situ via crosslinking to retain a specific and desired shape and to reduce the bioresorption rate. The pellet may be used in both open and minimally invasive surgical procedures and can be delivered either directly or via needle, catheter, trocar, or similar.

An embodiment is a pellet comprised of two polysaccharides: alginate and hyaluronate. The proportions of each polymer within the pellet may vary for different pellets/specimens. For example, the alginate component may constitute up to 95%, and no less than 10%, of the dry mass. In an embodiment, the pellets comprise no more than 75% and no less than 50% alginate by dry weight. In an embodiment, the pellets comprise between 60% and 70% alginate.

The alginate component is a copolymer of mannuronate (M) units and guluronate (G) chemical units. The alginate backbone consists of these two units arranged in repeating blocks and alternating blocks (e.g., MMMMMM, GGGGGG, and MGMGMG patterns). The proportion of M and G units in a particular alginate is dependent on the plant source from which the alginate is harvested. Typically, alginates are characterized by the proportion of M and G units. The alginate component in an embodiment may be any type of alginate including alginates with a high proportion of M units (i.e., high-M alginate), alginates with a high proportion of G units (i.e., high-G alginate) and blends of high-M and high-G alginates.

Alginates can be obtained in a variety of salt forms. The alginate salts of alkali metals (e.g., sodium and potassium) and magnesium are water soluble. The alginate salts of alkaline earth metals (e.g., calcium, strontium, barium) are water insoluble. Alginate can also form insoluble salts with transition metals such as iron and copper. The water insolubility of alginate salts is due to ionic crosslinking by multivalent cations of the G-units in alginate's backbone. An embodiment is a water-soluble alginate used to prepare solutions for molding. Preferably, sodium alginate is used for molding and subsequently converted to calcium alginate after the pellet is delivered into a patient. Calcium, an element found throughout the body, is an excellent choice of crosslinker from the point of view of biocompatibility.

Hyaluronate is an alternating polysaccharide of N-acetylglucosamine and glucuronic acid chemical units. The polymer can be obtained from both animal and bacterial sources and in several molecular weights. An acid form, hyaluronic acid, can be obtained but has limited water solubility. Hyaluronate stocks for research and clinical use are predominantly salts, particularly sodium salts. In an embodiment sodium hyaluronate salt is used for pellet preparation due to its commercial availability. Other salts can also be obtained, but unlike alginate, these salts are water soluble.

Hyaluronate is found throughout the connective tissues of the body particularly in the skin, cartilage, and vitreous fluid of the eye. It is an unusually large macromolecule that can reach molecular weights of up to several million. Hyaluronate is capable of binding to specialized proteins to form macromolecular complexes that are structural frameworks for tissue development and wound healing. The backbone of hyaluronate is highly negatively charged because of the heavy prevalence of carboxyl functionalities. Hyaluronate is unique in the body with the combination of high molecular weight and high charge density. These properties make hyaluronate capable of immobilizing significant volumes of water molecules to its surface, thereby helping tissues to maintain hydration and homeostasis.

Hyaluronate is highly conserved and exceptionally biocompatible. Hyaluronate is nearly ubiquitous throughout the tissues of the body; therefore, the immune system does not recognize it as foreign. Additionally, hyaluronate is strongly associated with wound healing and particularly with scar-free wound healing and fetal tissue development. For these reasons there has been much interest in the clinical application of hyaluronate to promote wound healing and regeneration.

An embodiment is a pellet comprised of both hyaluronate and alginate. In addition to these polymers the pellet can be hydrated to contain a significant proportion of water and can be classified as a hydrogel. Hydrogels are materials that immobilize water molecules onto their surface and often swell when exposed to large volumes of excess water. Hyaluronate is more hydrophilic than alginate and therefore hydrogel compositions with greater proportions of hyaluronate exhibit greater water swelling. At a molecular level, hydrogels are comprised of a network of polymer chains that are dispersed within an aqueous medium. Hydrogels tend to be biocompatible because water itself is biocompatible. Hydrogels therefore are attractive for clinical applications in which materials will come into close contact with living tissues. A feature of an embodiment is the ability to quickly hydrate the significantly desiccated pellet with an aqueous fluid.

Alginate can be crosslinked with calcium to form an insoluble mold. This crosslinked framework provides mechanical stability and shape to the hydrated pellet. The hyaluronate component is entrapped within the alginate gel and its release is limited by its large size compared to the pores of the alginate gel. A feature of an embodiment is the ability to mold the hydrogel in situ after placement onto complex, unique geometries. The crosslinked hydrogel takes on the shape of underlying and surrounding tissues.

Pellets are prepared through molding, flash freezing, and drying under vacuum. This preparation requires dissolving water-soluble salt forms of the alginate and hyaluronate in an aqueous mixture. Then a volume of the solution can be dispensed into a mold. A suitable mold can be of any shape or size such as beads, pads, or pellets. The water from the solution is rapidly frozen and sublimated from the mold to obtain a significantly desiccated pellet.

A more detailed discussion now follows.

### General

Hydrophilic long-chain biopolymers have long been employed in surgical anti-adhesion products intended to provide a gilding surface for target tissues and thereby limit unwanted postoperative soft tissue tethering. These biopolymers are typically high molecular weight and lend themselves to viscous solutions. Viscous solutions intended for surgical use are often prepared by first mixing reagents in aqueous solvents followed by water removal, to limit polymer degradation during sterilization processes. Lyophilization is a method for water removal from viscous, aqueous solutions. Lyophilization involves at least one freezing step followed by water sublimation under near-vacuum pressures. Resulting lyophilized materials are often brittle as water provides mechanical elasticity; removal of water reduces polymer rotation and flexibility. Lyophilized materials can be difficult or slow to rehydrate as polymer compression that occurs during freezing or dehydration limits subsequent access to water molecules during rehydration procedures. Furthermore, surgical delivery of viscous materials to target tissues is complicated by material loss, unintended placement, and delay of surgery. Of particular interest are biocompatible biopolymers that are easy to handle, are prepared for quick rehydration and use in an operating room setting, and can mold in situ onto challenging-to-reach or -to-visualize complex tissue geometries.

### Materials

Medical grade sodium alginate >200 kDa, G/M ratio ≥1.5 and medical grade sodium hyaluronate from Streptococcus equi. with molecular weight 1200-1900 kDa were purchased from IFF/Novamatrix (Sandvika, Norway). Water for injection, sterile USP grade (Corning Life Sciences, Tewksbury, MA, USA) was used. Methylene Blue (USP grade) was purchased from Biopharm Inc., (Hatfield, AR). All solvents were analytical grade. A customized 10-gauge blunt stainless-steel needle was purchased from Hamilton Company. All other materials such as syringes, microcentrifuge tubes, petri dishes, weigh boats were obtained from Fisher Scientific. A calcaneal goat tendon was obtained from a local butcher (Austin, TX).

### Experimental Section

Preparation of hyaluronic acid (HA) and sodium alginate (SA) solution blend: A 5.5% w/v solution of 80% sodium alginate and 20% sodium hyaluronate, in sterile water was stirred at 25° C. for 48 h to obtain a clear viscous solution. Viscosity was measured using a cone and plate DV1 viscometer (Ametek Brookfield, MA, USA) at 25° C. at a shear rate of 20 rpm.

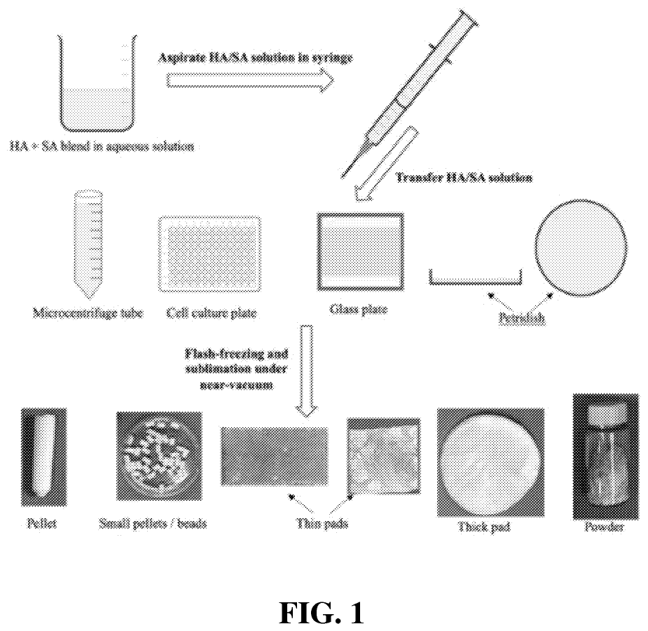

Preparation of HA/SA blended hydrogel matrix. The HA/SA hydrogel matrix was prepared using the freeze-drying technique as depicted in FIG. 1. HA/SA solution blend prepared immediately above was aspirated in 12 mL syringes. The HA/SA solution was aliquoted into containers suitable to obtain the desired shape, frozen, and lyophilized (−84° C., 0.0 mbar, 24 h).

Pellet: Two milliliters (2 mL) of HA/SA solution was transferred from the syringe using a 10-gauge blunt needle into a 2 mL microcentrifuge tube, followed by pre-freezing and lyophilization. Hydrogel pellets were prepared by pre-freezing the sample slowly at −18° C. for 8 h or rapidly over dry ice at −80° C. for 4 h.

Small pellets/beads: The HA/SA solution from the 12 mL syringe was used to fill a 96 well culture plate followed by freezing (−18° C., 8 h), and lyophilization to obtain small pellets/beads.

Pad: The HA/SA solution can be molded into pads of any size and thickness using a doctor blade. Thin pads of HA/SA solution were prepared using a doctor blade with 300 μm gap. Thicker pads were prepared by pouring a known volume of HA/SA solution in a square shaped weigh boat (2×2-in) or a petri dish (2.5-in diameter), followed by freezing (−18° C., 8 h), and lyophilization.

### Characterization of HA/SA Hydrogel

Chemical composition: HA-ELISA, a quantitative enzyme-linked immunosorbent assay, was conducted on a HA/SA hydrogel pellet solubilized in 1 mL of sterile water to determine the HA concentration. The assay was performed by Echelon Biosciences (Salt Lake City, Utah). HA competitive ELISA standard curve was generated using non-linear regression analysis with GraphPad Prism software. A sigmoidal dose response-variable slope curve (four parameter) analysis was utilized. The enzyme/substrate system was a colorometric assay comprised of alkaline phosphatase/pNPP phosphatase substrate. Absorbance measurements were read at 405 nm.

Hydration and viscosity: The prepared HA/SA hydrogel pellets were weighed and hydrated with 0.9% normal saline to match the starting volume prior to freezing. The solution was mixed with agitation at room temperature. Time to solubilize the lyophilized pellet to obtain a translucent homogenous gelatinous solution was recorded. The viscosity of the resulting solution (ca. 0.6 mL) was determined using a cone and plate DV1 viscometer (Ametek Brookfield, MA, USA) at 25° C. at a shear rate of 20 rpm. All measurements were performed in triplicate.

Structure (macroscopic) and morphology (microscopic): Lyophilized hydrogel samples were analyzed using high resolution optical microscopy (Keyence VHX-4000 series digital microscope) and scanning electron microscopy (SEM)/energy dispersive X-ray (EDX) spectroscopy (Tescan Vega S 3 LMU with EDAX Octane Plus EDX detector). An electron beam voltage of 15 kV and beam intensity of 15 was used for SEM-EDX under a high vacuum. Images of the sample surface and of the cross-section were collected.

Porosity and density: The porosity (ϵ) and the density (ρ) of the prepared hydrogel pellets were estimated by liquid displacement technique. Methanol (99.9%) was used in these measurements as a nonsolvent for HA and SA polymers constituting the hydrogel. The sample was weighted (W) and immersed in a graduated cylinder containing a specific volume of methanol (V1). After 5 min, the total liquid volume (V2) in the cylinder was recorded. The methanol-impregnated hydrogel pellet was removed from the cylinder, and the residual methanol volume (V3) was noted. Each sample was measured in triplicate. The porosity and the density of the hydrogel were expressed as follows: Porosity, ϵ(%)=(V1−V3)/(V2−V3)×100; Density (ρ)=W/(V2−V3)

Mechanical Tests: Mechanical properties such as hardness, toughness and stiffness were determined for the HA/SA hydrogel pellets at room temperature using a texture analyzer TA-XTplus (Stable Micro Systems, Surrey, UK) equipped with a 5 kg load cell and flat probe (TA-52, probe head diameter 7 mm) for compression. The tests were carried out at a compression speed of 5 mm/min up to 60% of strain. The stiffness was calculated from the slope of the curve in the linear region (strain from 0 % to 0.5%). Toughness, defined as the amount of energy absorbed per unit volume up to 60% of the sample strain, was calculated. Toughness is related to the area under the stress-strain curve. Hardness was also determined and is defined as the resistance of the studied material to mechanical penetration of an indenter or to a localized plastic deformation. Therefore, force to a set distance in the pellet was used to assess the hardness. The results were recorded by Texture Exponent software (version 6.1.5.0, Stable Micro Systems, Surrey, UK). All measurements were performed in triplicate.

Gel application and tissue encapsulation: An HA/SA lyophilized pellet was hydrated with 0.75 mL 0.9% normal saline in a syringe and mixed until a homogenous gelatinous solution was obtained. The gelatinous solution was applied as a thin layer onto the surface of a goat tendon. One (1) mL of 250 mM calcium solution mixed with a drop of methylene blue for visualization was used to crosslink the alginate on the tendon.

### Results:

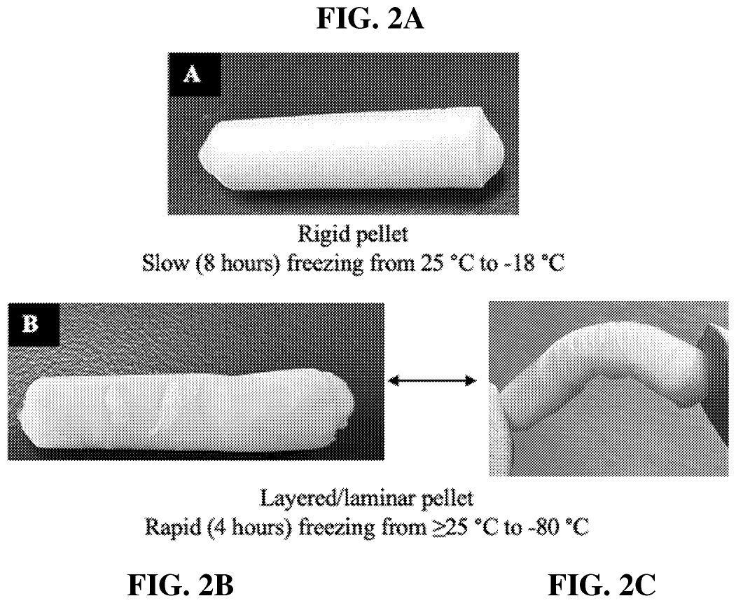

Preparation of HA/SA blended hydrogel matrix: Lyophilization of HA/SA polymer blended solution prepares hydrogels of any size or shape. Lyophilized hydrogels were prepared as pellets, pads, and beads of many sizes and thicknesses, or were milled into a powder (FIG. 1). Rapid cooling of the HA/SA solution from 25° C. or higher to −80° C. resulted in a flexible layered pellet structure whereas slowly cooling from 25° C. to −18° C. resulted in a uniform rigid pellet structure (FIGS. 2A-C). Specifically, FIGS. 2A-C show the effect of freezing conditions on the structure of the lyophilized pellets. FIG. 2A: Rigid pellet from a slow cooling rate (8 hours). FIGS. 2B-C: Layered or lamellar pellet from a rapid cooling rate (4 hours).

Chemical composition: The HA ELISA of a freeze-dried HA/SA hydrogel pellet with a mass of 123±2 mg confirmed that each pellet contained 20±1 mg of HA; the remaining mass, 103±1 mg, sodium alginate.

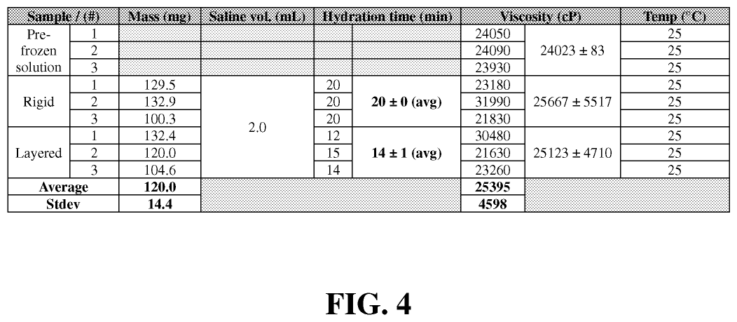

Hydration and viscosity: The rigid pellets completely hydrated in 2 mL saline after 20±0 minutes of exposure. The layered pellets completely hydrated in 2 mL saline after 14 ±1 minute of exposure (FIG. 4). The rate of hydration for the layered pellet was 30% faster than the rigid pellet.

The average viscosity of the pre-lyophilized solution was 24023±83 cP and that for the hydrated hydrogel was 25395±4598 cP (FIG. 4); no change in viscosity was observed between the solution prior to lyophilization and that of the hydrated lyophilized hydrogel. The average viscosity of the rigid hydrogel pellets was 25667±5517 cP and for layered pellets was 25123±4710 cP. Both rigid and layered pellets have comparable viscosities.

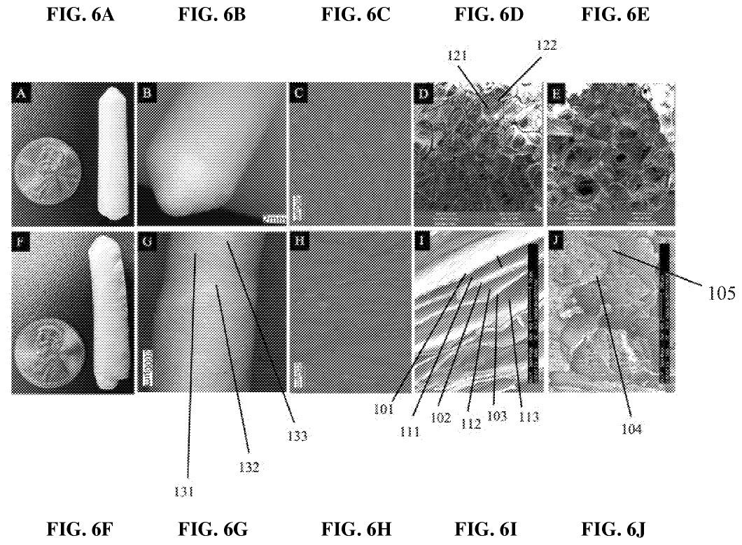

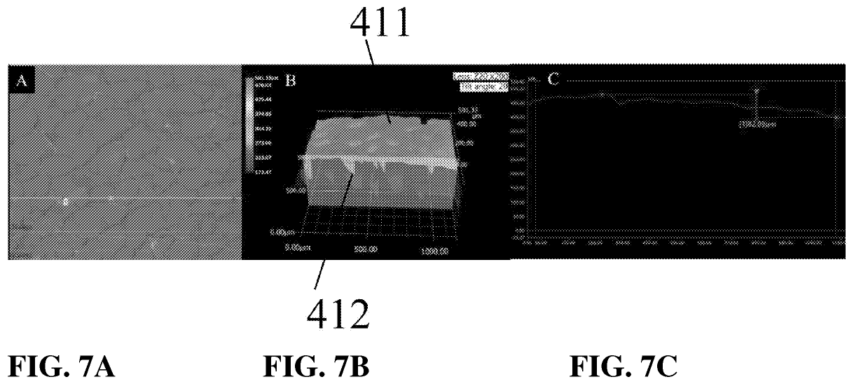

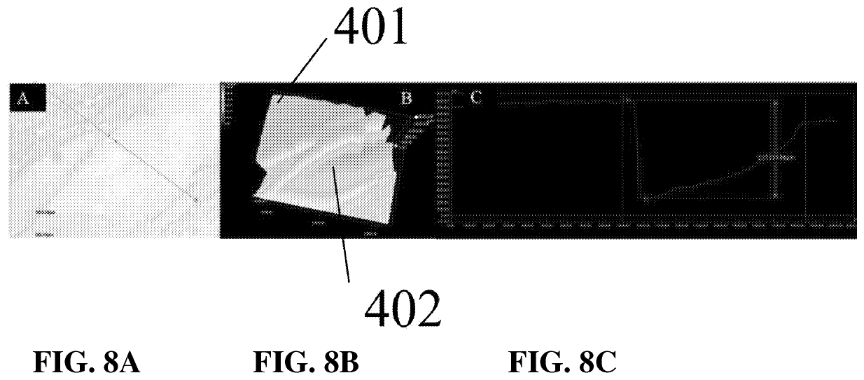

Structure and morphology: Pellets frozen slowly, over a period of 8 hours, from 25° C. to −18° C. resulted in a homogenous, rigid structure (FIGS. 6A-C). Microscopic images of the rigid pellet revealed an interconnective porous morphology (FIGS. 6D-E) and a uniform planar structure (FIGS. 7A-C). Pellets frozen rapidly, over a period of 4 hours, from ≥25° C. to −80° C. resulted in a layered or lamellar morphology (FIGS. 6F-J). Microscopic images of the lamellar structure reveal 400-1800 μm deep, parallel layers of polymer (FIG. 8A-C). Directional channels were observed in the layered pellet (FIGS. 6I-J).

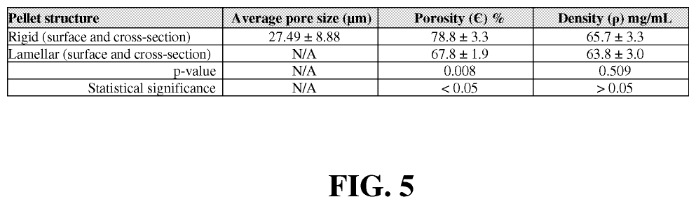

Porosity and Density: Microscopic images of the rigid structure reveal an average pore size of 26.58±10.82 μm at the sample surface and 28.50±6.57 μm at the sample cross-section, indicating a consistent bulk porosity (FIG. 5). These images also suggest high pore interconnectivity of 78.8±3.3 (ϵ) %. Microscopic images of the lamellar structure suggest directional channels resulting in 14% reduction of pore interconnectivity, 67.8±1.9 (ϵ) %, when compared to the rigid structure. No significant difference (p-value >0.05) in density was observed between the rigid and lamellar structures; the rigid and lamellar structures have comparable densities.

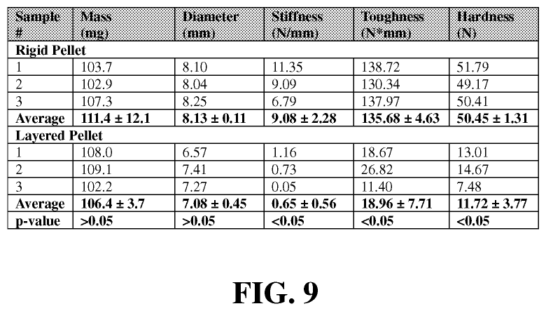

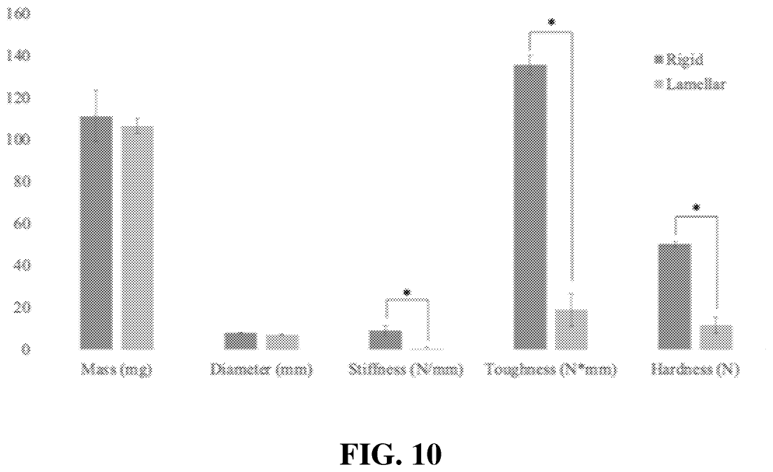

Mechanical Testing: The pellets compacted and flattened under compressive force, without fracture. The rigid pellets exhibited significantly higher stiffness, hardness, and toughness when compared to the lamellar pellets (p-value <0.05) (FIG. 9, FIG. 10). The lamellar pellets are 90% more flexible, 75% softer and 86% weaker when compared to the rigid pellets.

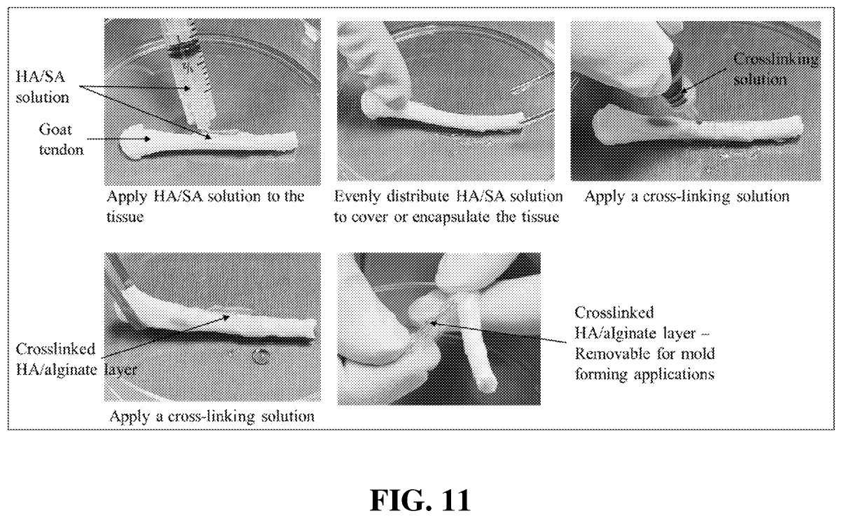

Gel application and tissue encapsulation: The hydrated HA/SA pellet (either rigid or lamellar) was easy to deliver via a syringe tip or a ≥20-gauge catheter. The hydrated HA/SA pellet can be applied and spread to form a thick or thin layer around the tissue. Following application of the hydrated pellet, application of crosslinking solution molded the HA/SA hydrogel into the applied shape and thickness (FIG. 11). The crosslinked hydrogel can then be removed for mold forming applications.

### Discussion

Dry hyaluronic acid/sodium alginate (HA/SA) hydrogels were prepared as pellets, beads, or pads. These hydrogels can be further milled to obtain a dried powder. Hydrogel preparation included the following steps: blending reagents in an aqueous solvent, molding into desired shape and size, freezing at various temperatures and duration, and then sublimating ice crystals under high-vacuum conditions. The macroscopic structure of the lyophilized hydrogel was shaped by a mold such as a microfuge tube or Petri dish. The microscopic structure of the lyophilized hydrogel was determined by ice crystal growth patterns created during freezing. Pellets with a homogenous rigid structure were obtained by slowly cooling from ≥25° C. to −18° C., over a period of 8 hours. Pellets with a layered or lamellar structure were obtained by rapidly cooling from ≥25° C. to −80° C., over a period of 4 hours. Homogenous structures occur in lyophilized materials prepared slowly, to give water molecules time to arrange themselves into hexagonal crystals, an energetically favored organization. Rapid cooling or flash-freezing induces supercooling in confined, discrete portions of the sample before crystal nucleation initiates and freezes via directional freezing. This directional freezing resulted in lamellar structures up to 1500 μm in depth (FIG. 8A-C) and directional channels in lyophilized pellets.

Rigid vs lamellar pellets had significantly different mechanical properties. Images of the rigid HA/SA pellet reveals a porous structure with irregular macropores and interconnectivity that is consistent throughout the bulk of the sample. Images of the lamellar HA/SA pellet reveals a layered structure with directional channels consistent throughout the bulk of the sample. The rigid pellets were significantly more porous than the layered pellets, however, the density was not statistically different. The sublimation process was held consistent for all samples, suggesting other attributes were responsible for these differences. The lamellar structure resulted in more flexible, softer, weaker pellets. These differences may be owed to the inability for water molecules to arrange themselves in an organized pattern during flash-freezing, thereby creating directional channels that are brittle and weaken the overall structure. Rapid cooling results in smaller ice crystals which effectively preserve the native structure of the polymer matrix; specifically, alginate layers because hydrophilic carboxyl groups face outwards towards moisture, and hydrophobic regions face inward, separating layers of carboxyl groups with water. This layered structure is maintained during rapid cooling and subsequent drying, leading to a lamellar structure. Slow cooling results in larger ice crystals which compress the surrounding polymer matrix, disrupting the native structure. These larger ice crystals result in a more porous structure. The interconnectivity of pores and compressed polymer matrix in the rigid structure may provide overall strength and ability to absorb energy before failure.

Despite the more brittle nature of the lamellar structure, the pellets were easily placed into syringes for rehydration. Lyophilized pellets with either rigid or lamellar structure, hydrated with saline, had comparable viscosity to the pre-lyophilized hydrogel solution. Several factors can influence hydration properties of a lyophilized sample including structure, surface area, homogeneity, and formation of channels. The hydration time for the rigid pellet was 30% slower when compared to the lamellar pellet. Slowly cooling the polymer blend solution may compress the adjacent polymers, resulting in thickened walls of the pores and decreasing available hydrophilic groups for protonation. Therefore, hydration may be prolonged. Instead of compressing polymers, the flash-freezing process preserves the lamellar structure of the alginate matrix positioning the hydrophilic groups for hydration. The lamellar pellets demonstrated accelerated hydration time owing to less compressed polymer chains that allowed water molecules to penetrate the matrix easily. Lamellar structures with channels offered greater surface area for the dissolution of the polymer.

The HA/SA hydrated solution can be crosslinked “on demand” to mold the viscous material in a desired shape and thickness. This capability increases the utility of the pellets for surgical application.

### Conclusions

Lyophilized HA/SA hydrogels were prepared as pellets, beads, pads, and a powder. Freezing conditions prior to lyophilization were reflected in the structure, mechanical properties, and rehydration rate of the resulting sample. Slowly freezing prepared rigid pellets with a homogenous, interconnected pore distribution, a compressed polymer matrix, and greater stiffness, hardness, and toughness. Flash-freezing prepared lamellar pellets with a homogenous, channeled pore distribution, layered polymer matrix, and lower stiffness, hardness, and toughness. Accelerated hydration time can be achieved with lamellar structured pellets; the pore channels and less compressed polymer matrix allow water molecules to penetrate the matrix easily; the lamellar structures provide greater surface area for dissolution of the polymer. The HA/SA hydrogel can be molded in situ with an external crosslinking stimulus.

### EXAMPLES

The following examples pertain to further embodiments.

Example 1. A hydrogel system comprising: a lyophilized hydrogel comprising alginate and hyaluronate; and a crosslinker; wherein the hydrogel has a lamellar morphology comprising parallel layers of the hydrogel.

For example, see layers 101, 102, 103 (i.e., lamellar) in FIG. 6I and layers 104, 105 in FIG. 6J of the relatively fast freezing process. This architecture is in contrast to the more porous nature of the relatively slow freezing process that results in pore or cell 121 surrounded by cell wall 122 in FIG. 6D. The fast-freezing process hydrogels are still porous, but they are statistically significantly less porous than the solid or rigid hydrogels.

Such an embodiment is distinguishable over conventional hydrogels because, in the least, the hydrogel contains hyaluronate (e.g., hyaluronic acid) and alginate (e.g., sodium alginate) and the hydrogel can be molded and crosslinked in situ to coat complex geometries. Further, the lamellar morphology allows for faster hydration and a non-compressed morphology that leads to a faster rate of bioresorption.

Embodiments may not necessarily include a crosslinker and may instead rely on, for example, crosslinking agents present in the patient's body. For example, calcium is abundant in the body and may supply ample crosslinking of the hydrogel without resorting to supplying an additional crosslinking agent to the hydrogel.

As used herein, “lyophilized” means a low temperature dehydration process that involves freezing the material and lowering pressure, thereby causing dehydration and removing ice by sublimation. The freezing may occur before the pressure is lowered. However, in some embodiments there may be some overlap between the freezing and pressure lowering steps.

A person of ordinary skill in the art can recognize whether a hydrogel is lyophilized by physical appearance. For example, a more hydrated hydrogel will be spongy or fibrous (i.e., stringy, hair-like, cotton candy-like, spider web-like) following lyophilization. However, a less hydrated hydrogel will be cakey or brittle following lyophilization.

A person of ordinary skill in the art may also recognize whether a hydrogel is lyophilized by the volume of water absorbed. For example, a hydrogel that is lyophilized will absorb a greater volume of water when compared to a hydrogel that was air-dried or solvent-dried. The air-dried hydrogel will absorb the second most water and the solvent-dried hydrogel will absorb the least volume of water. Swelling tests may be used to determine volume of water absorbed by a hydrogel.

A person of ordinary skill in the art may also recognize whether a hydrogel is lyophilized by examining the hydrogel's mechanical properties. For example, a hydrogel that is lyophilized will be more brittle than a hydrogel that is air-dried and less brittle than a hydrogel that is solvent-dried. For example, a texture analyzer can be used to measure stiffness, hardness, and/or toughness—all of which are dependent on the process used to dry the hydrogel (including the rate of cooling in the case of freeze-drying or rate of heating in the case of air-drying) as well as concentration of reagent, moisture content, molecular weight of the reagents.

As used herein, “desiccated” means dried or with little to no moisture. A person of ordinary skill in the art will recognize whether a hydrogel is desiccated by lack of water content (lyophilization is a method of desiccation, albeit extreme).

As used herein, “freeze-drying” is synonymous with lyophilizing.

As used herein, “sublimated” means transition of water from a solid state (ice) to a gaseous state (water vapor) without passing through the liquid phase.

As used herein, “lyophilized” and “desiccated” are not mutually exclusive terms and a hydrogel may be, for example, both “lyophilized” and “desiccated”.

Alternative version of example 1. A hydrogel system comprising: a hydrogel comprising alginate and hyaluronate; and a crosslinker; wherein the hydrogel has a lamellar morphology comprising parallel layers of the hydrogel.

Alternative version of example 1. A hydrogel system comprising: a desiccated hydrogel comprising alginate and hyaluronate; and a crosslinker; wherein the hydrogel has a lamellar morphology comprising parallel layers of the hydrogel.

Alternative version of example 1. A hydrogel system comprising: a freeze-dried hydrogel comprising alginate and hyaluronate; and a crosslinker; wherein the hydrogel has a lamellar morphology comprising parallel layers of the hydrogel.

Alternative version of example 1. A hydrogel system comprising: a sublimated hydrogel comprising alginate and hyaluronate; and a crosslinker; wherein the hydrogel has a lamellar morphology comprising parallel layers of the hydrogel.

Alternative version of Example 1. A hydrogel system comprising: a lyophilized hydrogel comprising alginate and hyaluronate; and a crosslinker; wherein the hydrogel has a lamellar morphology comprising layers of the hydrogel.

Alternative version of Example 1. A hydrogel system comprising: a lyophilized hydrogel comprising alginate and hyaluronate; and a crosslinker; wherein the hydrogel has a lamellar morphology comprising parallel layer portions of the hydrogel.

For example, the layer portion is a just a portion of the layer. The layer portion may include 20%, 40%, 60%, 80% or more of the layer.

Example 1.1. The hydrogel system of example 1, wherein the hydrogel includes channels that create crevasses or deep wedges into the bulk of the hydrogel such that at the surface or at a cross section there are deep ridges.

Example 1.2 The hydrogel system of example 1, wherein the hydrogel includes parallel hollow channels that are parallel to the parallel layers.

Example 1.3. The hydrogel system of example 1 comprising first and second parallel hollow channels, wherein: the parallel layers include first (101), second (102), and third (103) layers; the first channel (111) is between the first and second layers; the second channel (112) is between the second and third layers; no other layer is between the first and second layers; no other layer is between the second and third layers.

See FIG. 6I.

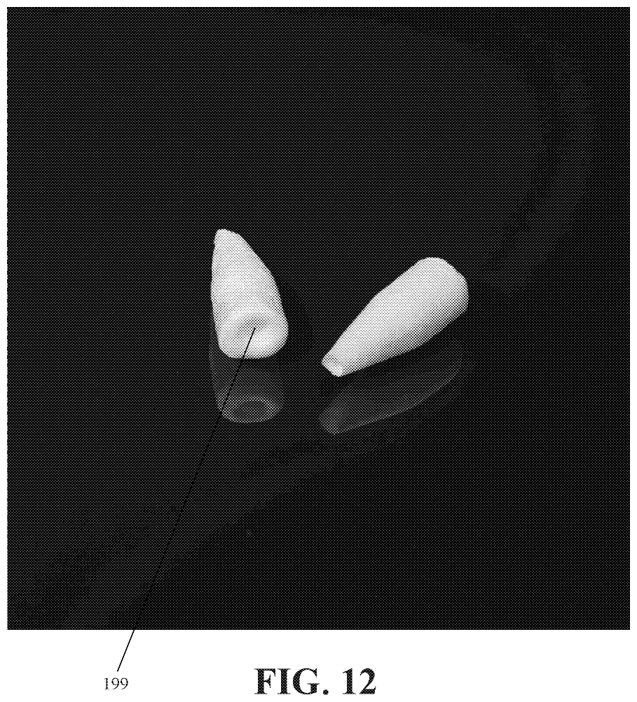

As used herein, “parallel” does not require the entire layer be perfectly parallel with another layer. For example, FIG. 6I illustrates “a lamellar morphology comprising parallel layers of the lyophilized hydrogel”. Further, FIG. 8B shows parallel layers. “Parallel layers” do not necessarily mean “parallel planes” but may instead include parallel curved surfaces that maintain a relatively constant distance between each other for some distance or length of the surface (i.e., the entire layer does not need to maintain the same distance away from an immediately adjacent layer). For example, see FIG. 12, which shows an embodiment having a circular cross-section and a somewhat frustoconical general form. The embodiment of FIG. 12 still has parallel layers albeit curved (at least partially) about void 199. Regarding void 199, the pellet/hydrogel may include a void 199 that may pass only partially into the pellet or may traverse the entire pellet such that there are multiple openings to the void collectively at opposing ends of the pellet. The central void (or, more generally, void or voids) helps speed hydration of the hydrogel by increasing surface area of the hydrogel that is exposed to a reconstituting medium (e.g., saline).

Another version of Example 1.3. The hydrogel system of example 1 comprising first and second hollow channels, wherein: the layers include first (101), second (102), and third (103) layers; the first channel (111) is between the first and second layers; the second channel (112) is between the second and third layers; no other layer is between the first and second layers; no other layer is between the second and third layers.

Example 1.4 The hydrogel system according to any examples 1-1.3, wherein: the hydrogel includes a void between two of the parallel layers; no additional layer is between the two parallel layers.

Such a void includes the aforementioned channels. The void allows for a non-compressed morphology that leads to faster rehydration and a faster rate of bioresorption. The void may extend from an outer surface of the hydrogel and extend into the hydrogel forming a crevasse. The crevasses may form a deep wedge into the hydrogel that does not go all the way through the hydrogel but instead creates deep ridges. At any point either on the surface of the hydrogel or a cross section of the hydrogel a deep cut, crevasse, or channel may be visible. As mentioned above, such a channel or void may extend 400 to 1800 microns deep into the hydrogel from an outer surface of the hydrogel.

Example 2. The hydrogel system of example 1.4, wherein: the void has a depth between 400 and 1900 microns; the depth starts from an outer surface of the hydrogel and extends into the hydrogel.

Example 2.1 The hydrogel system of example 2, wherein the depth extends into the hydrogel and orthogonal to the outer surface of the hydrogel.

In various embodiments the depth is 300, 500, 800, 1000, 1200, 1400, 1600, 1800 microns. See, e.g., the blue depth of FIG. 8B (area 402). See also FIG. 8C.

Example 3. The hydrogel system of example 1, wherein the hydrogel is a powder.

Example 4. The hydrogel system according to any of examples 1-2, wherein the hydrogel does not contain collagen.

Example 5. The hydrogel system according to any of examples 1-3, wherein the hydrogel consists essentially of alginate and hyaluronate.

As used herein, “consists essentially” means the hydrogel includes only the alginate and the hyaluronate and nothing else beyond trace amounts of other materials.

Example 6. The hydrogel system according to any of examples 1-4 comprising: a plurality of hydrogels each comprising alginate and hyaluronate; a delivery conduit including the hydrogel and the plurality of hydrogels.

Example 7. The hydrogel system of example 5, wherein the delivery conduit includes at least one a syringe, a needle, a catheter, or combinations thereof.

Example 8. The hydrogel system according to any of examples 1-6, wherein: the alginate constitutes no less than 10% of dry mass of the hydrogel; the hyaluronate constitutes no less than 50% of the dry mass of the hydrogel.

Example 9. The hydrogel system according to any of examples 1-7 comprising sodium alginate, wherein the alginate is included within the sodium alginate.

Example 10. The hydrogel system according to any of examples 1-8, wherein the hydrogel is primarily un-crosslinked.

Example 11. The hydrogel system according to any of examples 1 or 3-9, wherein the hydrogel is a pellet with a non-planar form.

Example 12. The hydrogel system according to any of examples 1 or 3-10, wherein the hydrogel has an ovular cross-section.

As used herein, an oval includes various forms such as a circular form (e.g., when its major and minor axes equal one another), egg-like form (e.g., when its major and minor axes do not equal one another), and the like.

Example 13. The hydrogel system according to any of examples 1-11, wherein the hydrogel is desiccated.

Example 14. The hydrogel system according to any of examples 1-12, wherein each of the layers of polymer is between 400-1800 μm in thickness.

Example 15. The hydrogel system according to any of examples 1-13, wherein the crosslinker includes calcium.

The amount of crosslinker (e.g., calcium) may vary for different embodiments. For example, a planar hydrogel embodiment may include more or less crosslinker than a hydrogel having a frusto-conical shape. The crosslinker amount may help fine tune hydration times for hydrogels of varying physical forms.

Example 16. The hydrogel system according to any of examples 1-14, wherein the hydrogel has an outer surface that is striated.

See, for example, striations 131, 132, 133 of FIG. 6G.

For example, FIGS. 6G and 6I illustrate “an outer surface that is striated”. In contrast, FIGS. 6C and 6D are not striated but instead show porous interconnected cells.

Example 17.The hydrogel system according to any of examples 1-15, wherein the hydrogel has a stiffness (N/mm) of less than 1.0.

However, in other embodiments the stiffness is less than 1.5, 1.3, 0.8, 0.5.

Example 18. The hydrogel system according to any of examples 1-16, wherein the hydrogel has a toughness (N*mm) of less than 30.00.

However, in other embodiments the toughness is less than 50.00, 40.00, 20.00, 10.00.

Example 19. The hydrogel system according to any of examples 1-17, wherein the hydrogel has a hardness (N) of less than 20.00.

However, in other embodiments the toughness is less than 25.00, 15.00, 10.00.

Example 21. The hydrogel system of example 3 comprising third and fourth parallel hollow channels, wherein: the parallel layers include fourth and fifth layers; the third channel is between the first and fourth layers; the fourth channel is between the third and fifth layers; no other layer is between the first and fourth layers; no other layer is between the third and fifth layers.

Example 22. The hydrogel system of example 21 comprising first and second outermost surfaces that oppose one another and that are coupled to one another by a side wall, wherein: the void is included in the side wall; the first surface outermost surface includes the fourth layer.

Example 23. The hydrogel system of example 21 comprising first and second outermost surfaces that oppose one another and that are coupled to one another by a side wall, wherein: the void is included in the side wall; the first, second, third, fourth, and fifth layers are collectively between the first and second outermost surfaces; the first outermost surface includes none of the first, second, third, fourth, or fifth layers; the second outermost surface includes none of the first, second, third, fourth, or fifth layers.

Example 1a. A method comprising: providing the hydrogel according to any of examples 1-23; hydrating the hydrogel; contacting a patient's tissue with the hydrogel.

For example, a user may hydrate the freeze-dried pellet with saline or a drug mixture, etc. in a syringe system prior to implanting. Once the pellet is dissolved, the user may deliver the gel to the patient's target tissues, such as tissues that have complex geometry. Then, once the viscous gel is delivered, the surgeon may crosslink the gel in place, creating a crosslinked, mechanically stable hydrogel with geometry unique to the patient (i.e., precise dimensions). This hydrogel then is a mold of the patient's unique tissues (e.g., for tumor removal areas that need to be reconstructed or for prosthetics). Alternatively, the gel may be delivered with mild or no crosslinking and can remain in place as a drug delivery device or to prevent tissues from sticking to one another. The gel may crosslink due to, for example, calcium within the tissue but such crosslinking would be slower and less thorough than if, for example, the surgeon applies a calcium crosslinking agent from a kit or otherwise not already present in the patient.

Example 2a. The method of example 1a comprising hydrating the hydrogel before contacting the patient's tissue with the hydrogel.

Example 2.1a The method of example 2a comprising hydrating the hydrogel while the hydrogel is in a container.

Example 2.2a The method of example 2.1a comprising dispensing the hydrated hydrogel from the container directly onto the patient's tissue.

Example 2.3a The method of example 2.2a, wherein the container includes at least one of a syringe or a needle.

Example 3a. The method of example 1a comprising hydrating the hydrogel after contacting the patient's tissue with the hydrogel.

Example 4a. The method according to any of examples 1a-3a comprising applying the crosslinker to the hydrogel after contacting the patient's tissue with the hydrogel.

Example 5a. The method according to any of examples 1a-3a comprising applying the crosslinker to the hydrogel before contacting the patient's tissue with the hydrogel.

Example 6a. The method according to any of examples 4a-5a comprising molding the hydrogel around the patient's tissue after applying the crosslinker to the hydrogel.

Example 6.1a. The method according to any of examples 1a-8a comprising statically fixing the hydrogel into a three-dimensional form in response to molding the hydrogel around the patient's tissue after applying the crosslinker to the hydrogel.

Example 7a. The method according to any of examples 1a-6.1a comprising: determining an amount of the crosslinker to apply to the hydrogel; applying the determined amount of the crosslinker to the hydrogel in response to determining the amount of the crosslinker to apply to the hydrogel; adjusting a bioresorption rate of the hydrogel based on the determined amount of the crosslinker applied to the hydrogel.

Example 8a. The method according to any of examples 1a-7a comprising: determining an amount of hydrogel needed to contact the patient's tissue; in response to determining the amount of hydrogel needed to contact the patient's tissue, selecting a subset of a plurality of hydrogels each comprising alginate and hyaluronate; applying the subset of the plurality of hydrogels to the patient's tissue.

Example 1b. A method comprising creating the hydrogel according to any examples 1-23.

Example 2b. The method of example 1b comprising: blending sodium hyaluronate and sodium alginate to form an aqueous solution; aspirating the aqueous solution into a container; freezing the aqueous solution while the aqueous solution is in the container; lowering the pressure in the container to a pressure less than atmospheric pressure.

The freezing may occur before the pressure is lowered. However, in some embodiments there may be some overlap between the freezing and pressure lowering steps.

Example 3b. n/a

Example 4b. The method according to any of examples 1b-2b comprising lyophilizing the aqueous solution.

Example 5b. The method according to any of examples 1b-4b comprising molding the hydrogel into a form that conforms to a least a portion of the container.

Example 6b. The method according to any of examples 1b-5b comprising desiccating the aqueous solution.

Example 7b. The method according to any of examples 1b-6b comprising sublimating the aqueous solution while the aqueous solution is in the container and under a pressure less than atmospheric pressure.

Example 8b. The method according to any of examples 1b-7b comprising freezing the aqueous solution for 5 hours or less in an environment that is colder than −50° C..

However, in another embodiment the time period is between 2 and 4 hours at temperature between −35 and −50° C.. In another embodiment the time period is between 2 and 4 hours at temperature between −65 and −90° C..

Example 9b. The method of example 8b wherein the aqueous solution is less than 5 ml.

Example 1c. A hydrogel system comprising: a lyophilized hydrogel comprising alginate and hyaluronate; and a crosslinker; wherein the hydrogel has a lamellar morphology comprising parallel layers of the hydrogel.

Alternative version of Example 1c. 1. A hydrogel system comprising: a lyophilized hydrogel comprising alginate and hyaluronate; and a crosslinker; wherein the hydrogel has a lamellar morphology comprising layers of the hydrogel.

Alternative version of Example 1c. A hydrogel system comprising: a lyophilized hydrogel comprising alginate and hyaluronate; and a crosslinker; wherein the hydrogel has a lamellar morphology comprising parallel layer portions of the hydrogel.

Example 2c. The hydrogel system of example 1c comprising first and second parallel hollow channels, wherein: the parallel layers include first (101), second (102), and third (103) layers; the first channel (111) is between the first and second layers; the second channel (112) is between the second and third layers; no other layer is between the first and second layers; no other layer is between the second and third layers.

Example 3c. The hydrogel system according to any examples 1c-2c, wherein: the hydrogel includes a void between two of the parallel layers; no additional layer is between the two parallel layers.

Example 4c. The hydrogel system of example 3c, wherein: the void has a depth between 400 and 1900 microns; the depth starts from an outer surface of the hydrogel and extends into the hydrogel.

Example 5c. The hydrogel system of example 4c, wherein the depth extends into the hydrogel and orthogonal to the outer surface of the hydrogel.

Example 6c. The hydrogel system according to any of examples 1c-5c, wherein the hydrogel does not contain collagen.

Example 7c. The hydrogel system according to any of examples 1c-6c, wherein the hydrogel consists essentially of alginate and hyaluronate.

Example 8c. The hydrogel system according to any of examples 1c-7c comprising: a plurality of hydrogels each comprising alginate and hyaluronate; a delivery conduit including the hydrogel and the plurality of hydrogels.

Example 9c. The hydrogel system of example 8c, wherein the delivery conduit includes at least one a syringe, a needle, a catheter, or combinations thereof.

Example 10c. The hydrogel system according to any of examples 1c-9c, wherein: the alginate constitutes no less than 10% of dry mass of the hydrogel; the hyaluronate constitutes no less than 50% of the dry mass of the hydrogel.

Example 11c. The hydrogel system according to any of examples 1c-10c comprising sodium alginate, wherein the alginate is included within the sodium alginate.

Example 12c. The hydrogel system according to any of examples 1c-11c, wherein the hydrogel is primarily un-crosslinked.

Example 13c. The hydrogel system according to any of examples 1c-12c, wherein the hydrogel is a pellet with a non-planar form.

Example 14c. The hydrogel system according to any of examples 1c-13c, wherein the hydrogel has an ovular cross-section.

Example 15c. The hydrogel system according to any of examples 1c-14c, wherein the hydrogel is desiccated.

Example 16c. The hydrogel system according to any of examples 1c-15c, wherein each of the layers of polymer is between 400-1800 μm in thickness.

Example 17c. The hydrogel system according to any of examples 1c-16c, wherein the crosslinker includes calcium.

Example 18c. The hydrogel system according to any of examples 1c-17c, wherein the hydrogel has an outer surface that is striated.

Example 19c. The hydrogel system according to any of examples 1c-18c, wherein the hydrogel has a stiffness (N/mm) of less than 1.0.

Example 20c. The hydrogel system according to any of examples 1c-19c, wherein the hydrogel has a toughness (N*mm) of less than 30.00.

Example 21c. The hydrogel system according to any of examples 1c-20c, wherein the hydrogel has a hardness (N) of less than 20.00.

Example 22c. A method comprising: providing the hydrogel according to any of examples 1c-21c; hydrating the hydrogel; contacting a patient's tissue with the hydrogel.

Example 23c. The method of example 22c comprising hydrating the hydrogel before contacting the patient's tissue with the hydrogel.

Example 24c. The method of example 23c comprising hydrating the hydrogel while the hydrogel is in a container.

Example 25c. The method of example 24c comprising dispensing the hydrated hydrogel from the container directly onto the patient's tissue.

Example 26c. The method according to any of examples 24c-25c, wherein the container includes at least one of a syringe or a needle.

Example 27c. The method of example 22c comprising hydrating the hydrogel after contacting the patient's tissue with the hydrogel.

Example 28c. The method according to any of examples 22c-27c comprising applying the crosslinker to the hydrogel after contacting the patient's tissue with the hydrogel.

Example 29c. The method according to any of examples 22c-27c comprising applying the crosslinker to the hydrogel before contacting the patient's tissue with the hydrogel.

Example 30c. The method according to any of examples 22c-29c comprising molding the hydrogel around the patient's tissue after applying the crosslinker to the hydrogel.

Example 31c. The method of example 30c comprising statically fixing the hydrogel into a three-dimensional form in response to molding the hydrogel around the patient's tissue after applying the crosslinker to the hydrogel.

Example 32c. The method according to any of examples 22c-31c comprising: determining an amount of the crosslinker to apply to the hydrogel; applying the determined amount of the crosslinker to the hydrogel in response to determining the amount of the crosslinker to apply to the hydrogel; adjusting a bioresorption rate of the hydrogel based on the determined amount of the crosslinker applied to the hydrogel.

Example 33c. The method according to any of examples 22c-32c comprising: determining an amount of hydrogel need to contact the patient's tissue; in response to determining the amount of hydrogel needed to contact the patient's tissue, selecting a subset of a plurality of hydrogels each comprising alginate and hyaluronate; applying the subset of the plurality of hydrogels to the patient's tissue.

Example 34c. A method comprising creating the hydrogel according to any examples 1c-21c.

Example 35c. The method of example 34c comprising: blending sodium hyaluronate and sodium alginate to form an aqueous solution; aspirating the aqueous solution into a container; freezing the aqueous solution while the aqueous solution is in the container; lowering the pressure in the container to a pressure less than atmospheric pressure.

Example 36c. The method of example 35c comprising lyophilizing the aqueous solution.

Example 37c. The method according to any of examples 35c-36c comprising molding the hydrogel into a form that conforms to a least a portion of the container.

Example 38c. The method according to any of examples 35c-37c comprising desiccating the aqueous solution.

Example 39c. The method according to any of examples 35c-38c comprising sublimating the aqueous solution while the aqueous solution is in the container and under a pressure less than atmospheric pressure.

Example 40c. The method according to any of examples 35c-39c comprising freezing the aqueous solution for 5 hours or less in an environment that is colder than −50° C.

Example 41c. The method according to any of examples 35c-40c wherein the aqueous solution is less than 5 ml.

The foregoing description of the embodiments of the invention has been presented for the purposes of illustration and description. It is not intended to be exhaustive or to limit the invention to the precise forms disclosed. This description and the claims following include terms, such as left, right, top, bottom, over, under, upper, lower, first, second, etc. that are used for descriptive purposes only and are not to be construed as limiting. For example, terms designating relative vertical position refer to a situation where a side of a substrate is the “top” surface of that substrate; the substrate may actually be in any orientation so that a “top” side of a substrate may be lower than the “bottom” side in a standard terrestrial frame of reference and still fall within the meaning of the term “top.” The term “on” as used herein (including in the claims) does not indicate that a first layer “on” a second layer is directly on and in immediate contact with the second layer unless such is specifically stated; there may be a third layer or other structure between the first layer and the second layer on the first layer. The embodiments of a device or article described herein can be manufactured, used, or shipped in a number of positions and orientations. Persons skilled in the relevant art can appreciate that many modifications and variations are possible in light of the above teaching. Persons skilled in the art will recognize various equivalent combinations and substitutions for various components shown in the Figures. It is therefore intended that the scope of the invention be limited not by this detailed description, but rather by the claims appended hereto.

1. A method comprising:
blending sodium hyaluronate and sodium alginate to form an aqueous solution;
moving the aqueous solution into a container;
freezing the aqueous solution while the aqueous solution is in the container;
lowering a pressure in the container to a pressure less than atmospheric pressure;
forming a hydrogel in response to freezing the aqueous solution while the aqueous solution is in the container and lowering the pressure in the container to less than atmospheric pressure.

2. The method of claim 1, wherein the hydrogel has a lamellar morphology.

3. The method of claim 2, wherein the hydrogel comprises parallel layers of the hydrogel.

4. The method of claim 1 comprising freezing the aqueous solution while the aqueous solution is in the container and before lowering the pressure in the container to less than atmospheric pressure.

5. The method of claim 4 comprising lyophilizing the aqueous solution.

6. The method of claim 4 comprising molding the hydrogel into a form that conforms to a least a portion of the container.

7. The method of claim 6 comprising desiccating the aqueous solution.

8. The method of claim 4 comprising sublimating the aqueous solution while the aqueous solution is in the container which is at less than atmospheric pressure.

9. The method of claim 4 comprising freezing the aqueous solution for 5 hours or less in an environment that is colder than −50° C..

10. The method of claim 9 wherein the aqueous solution is less than 5 ml.

11. The method of claim 1 comprising freezing the aqueous solution while the aqueous solution is in the container and while lowering the pressure in the container to less than atmospheric pressure.

12. The method of claim 11 comprising lyophilizing the aqueous solution.

13. The method of claim 11 comprising molding the hydrogel into a form that conforms to a least a portion of the container.

14. The method of claim 13 comprising desiccating the aqueous solution.

15. The method of claim 11 comprising sublimating the aqueous solution while the aqueous solution is in the container which is at less than atmospheric pressure.

16. The method of claim 11 comprising freezing the aqueous solution for 5 hours or less in an environment that is colder than −50° C..

17. The method of claim 16 wherein the aqueous solution is less than 5 ml.

18. The method of claim 1 wherein the aqueous solution includes about a 5% w/v solution of about 80% sodium alginate and about 20% sodium hyaluronate in sterile water.

19. The method of claim 1 wherein the aqueous solution has a viscosity between about 23000 and about 26000 cP.

20. The method of claim 1 comprising hydrating the hydrogel, wherein the hydrated hydrogel has a viscosity between about 20,000 to 30,000 cP.

21. The method of claim 1, wherein:
the hydrogel includes alginate and hyaluronate;
the alginate is at least 10% and nor more than 95% of a dry mass of the hydrogel.

22. The method of claim 21, wherein the hydrogel consists essentially of the alginate and the hyaluronate.

23. The method of claim 1, wherein:
the hydrogel includes alginate and hyaluronate;
the alginate is at least 50% and no more than 75% of a dry mass of the hydrogel.

24. The method of claim 1, wherein:
the hydrogel includes alginate and hyaluronate;
the alginate is at least 60% and no more than 70% of a dry mass of the hydrogel.

25. The method claim 1 comprising forming the hydrogel in a pellet shape.

26. The method or claim 1 wherein:
the container includes an arcuate surface;
the hydrogel is in pellet form in response to the container including an arcuate surface.

27. The method of claim 1, wherein the hydrogel consists essentially of alginate and hyaluronate.
